(19) United States
(12) Patent Application Publication
(10) Pub. No.: US 2024/0400481 A1
(43) Pub. Date: Dec. 5, 2024

(54) USE OF AN OFF GAS COMPRISING HYDROGEN FOR METHANOL PRODUCTION

(71) Applicant: SABIC GLOBAL TECHNOLOGIES B.V., Bergen op Zoom (NL)

(72) Inventors: Mubarik Ali Bashir, Houston, TX (US); Tjeerd Alexander Nijhuis, Roermond (NL); Christian Okolo, Roermond (NL); Uma K. Sankar, Bangalore, Karnataka (IN); Muammar Mohammad Al-Ansari, Jubail (SA); Srikant Vasant Badgandi, Bangalore, Karnataka (IN)

(21) Appl. No.: 18/700,480

(22) PCT Filed: Oct. 11, 2022
(86) PCT No.: PCT/IB2022/059745

§ 371 Date: Apr. 11, 2024

(30) Foreign Application Priority Data
Oct. 12, 2021 (EP) 21202195.0

(51) Int. Cl. C07C 29/151

(52) U.S. Cl. CPC C07C 29/1518

(57) ABSTRACT

A method for producing methanol includes feeding a synthesis gas stream to a methanol production assembly; feeding an off gas stream including hydrogen to the methanol production assembly; feeding a CO₂ stream to the methanol production assembly; and reacting the synthesis gas stream and the CO₂ stream in the methanol production assembly to produce methanol, wherein the method further includes reacting the off gas stream with the synthesis gas stream and the CO₂ stream in the methanol production assembly to produce methanol, or feeding a reforming feed stream and the off gas stream including hydrogen to a reforming portion of the methanol production assembly to form the synthesis gas stream.

### BACKGROUND

Methanol is a highly versatile chemical used in many areas of the chemical industry. For example, methanol can be used as feedstock in the manufacture of various chemicals, including plastics, paints, plywood, biodiesels, and textiles. Furthermore, methanol can also be used as a denaturant, a solvent, and an antifreeze reagent. Further yet, many specialized vehicles have been developed to consume methanol as an alternative fuel either in combination with gasoline or alone.

### BRIEF DESCRIPTION

While some conventional methods for producing methanol using certain gases exist, it is noted that opportunities for improvement exist and are addressed by the processes and methods of the present disclosure. An off gas stream including hydrogen produced in a chemical or molecule production system may be used as fuel (e.g., burned) in the system. Such an exemplary off gas stream can include 70 to 100 mole percent (mol %) hydrogen, for example, 70 to less than 100 mol % hydrogen, 70 to 99 mol % hydrogen, 70 to 95 mol % hydrogen, 70 to 90 mol % hydrogen, or 80 to 90 mol % hydrogen, based on a total number of moles of the off gas stream. However, the present inventors discovered and the present disclosure advantageously provides that use of such an off gas stream to produce methanol can provide an advantageous and more productive use of such an off gas stream and reduce anthropogenic carbon dioxide (CO₂) emission into the atmosphere.

The present disclosure provides a method of utilizing an off gas stream including hydrogen as a feed for methanol production with CO₂ as a raw material or as a feed for formation of synthesis gas to be used in methanol production. Additional methanol can be produced with a same amount of natural gas consumption and overall greenhouse gas emission reduction (e.g., reduction of CO₂ and nitrogen oxides (NOx)) as compared to a method that does not utilize an off gas stream including hydrogen.

Disclosed, in various embodiments, are processes and methods for producing methanol.

Disclosed herein is a method for producing methanol including feeding a synthesis gas stream to a methanol production assembly; feeding an off gas stream including hydrogen to the methanol production assembly; feeding a CO₂ stream to the methanol production assembly; and reacting the synthesis gas stream and the CO₂ stream in the methanol production assembly to produce methanol, wherein the method further includes reacting the off gas stream with the synthesis gas stream and the CO₂ stream in the methanol production assembly to produce methanol, or feeding a reforming feed stream and the off gas stream to a reforming portion of the methanol production assembly to form the synthesis gas stream.

Disclosed herein is an integrated method for producing methanol including feeding a synthesis gas stream to a synthesis portion of a methanol production assembly; feeding an off gas stream including hydrogen from an off gas source to the methanol production assembly; feeding a CO₂ stream to the synthesis portion of the methanol production assembly; providing an auxiliary natural gas stream from a reforming portion of the methanol production assembly to the off gas source; and reacting the synthesis gas stream and the CO₂ stream in the synthesis portion of the methanol production assembly to produce methanol, wherein the method further includes reacting the off gas stream with the synthesis gas stream and the CO₂ stream in the synthesis portion of the methanol production assembly to produce methanol, or feeding a reforming feed stream and the off gas stream to the reforming portion of the methanol production assembly.

Disclosed herein is an integrated method for producing methanol including feeding a natural gas feed stream to a reforming portion of a methanol production assembly; separating the natural gas feed stream into at least a reforming feed stream, a natural gas fuel stream, and an auxiliary natural gas stream; feeding the reforming feed stream to a reforming reactor to form a synthesis gas stream; using the natural gas fuel stream as fuel to provide heat to the reforming reactor; providing the auxiliary natural gas stream to a chemical or molecule production system to produce a chemical or molecule; forming an off gas stream including hydrogen in the chemical or molecule production system; feeding the synthesis gas stream to a synthesis portion of the methanol production assembly; feeding the off gas stream from the chemical or molecule production system to the methanol production assembly; feeding a CO₂ stream to the synthesis portion of the methanol production assembly; and reacting the synthesis gas stream and the CO₂ stream in the synthesis portion of the methanol production assembly to produce methanol, wherein the method further includes reacting the off gas stream with the synthesis gas stream and the CO₂ stream in the synthesis portion of the methanol production assembly to produce methanol, or feeding the off gas stream from the chemical or molecule production system to the reforming reactor in which synthesis gas stream is formed.

The above described and other features are exemplified by the following figures and detailed description.

Any combination or permutation of embodiments is envisioned. Additional advantageous features, functions and applications of the disclosed processes and methods of the present disclosure will be apparent from the description which follows, particularly when read in conjunction with the appended figures.

### BRIEF DESCRIPTION OF THE DRAWINGS

The following figures are exemplary embodiments wherein the like elements are numbered alike.

FIG. 1 is a diagram of an embodiment for utilizing off gas including hydrogen for methanol production;

FIG. 2 is a diagram of an embodiment for utilizing off gas including hydrogen for methanol production;

FIG. 3 is a diagram of an embodiment for utilizing CO₂ and off gas including hydrogen for methanol production;

FIG. 4 is a schematic overview of a process model used in the examples;

FIG. 5 is a schematic overview of a process model used in the examples; and

FIG. 6 is a schematic overview of a process model used in the examples.

### DETAILED DESCRIPTION

The exemplary embodiments disclosed herein are illustrative of advantageous methods for producing methanol. It should be understood, however, that the disclosed embodiments are merely exemplary of the present disclosure, which may be embodied in various forms. Therefore, details disclosed herein with reference to exemplary methods for producing methanol are not to be interpreted as limiting, but merely as the basis for teaching one skilled in the art the advantageous methods for producing methanol of the present disclosure.

The disclosed method utilizes an off gas stream including hydrogen to produce methanol. Use of the off gas stream including hydrogen to produce methanol can result in valorization of the off gas stream, which may otherwise be used as fuel (e.g., burned), and provide increased economic benefit. Natural gas may be used as fuel in place of off gas in the disclosed method, with natural gas consumption less than or equal to natural gas consumption in a method that does not utilize an off gas stream to produce methanol. The off gas stream can include 70 to 100 mole percent (mol %) hydrogen, preferably 70 to less than 100 mol % hydrogen, 70 to 99 mol % hydrogen, 70 to 95 mol % hydrogen, 70 to 90 mol % hydrogen, or 80 to 90 mol % hydrogen, based on a total number of moles of the off gas stream. In an embodiment, the off gas stream can be used without treatment (e.g., purification) as a feed for methanol production. In an embodiment, the off gas stream can be treated (e.g., purified) before being used as a feed for methanol production.

As used herein, as readily understood by one of ordinary skill in the art, the term “off gas” refers to a gaseous by-product of a chemical or molecule production process, in contrast to a “purge stream,” which can refer to a stream in which material, such as unreacted gas, is controllably removed from a chemical or molecule production process, for example, to prevent accumulation of an undesired material. An off gas stream can include-without being subject to separation-70 to 100 mol % hydrogen, preferably 70 to less than 100 mol % hydrogen, 70 to 99 mol % hydrogen, 70 to 95 mol % hydrogen, 70 to 90 mol % hydrogen, or 80 to 90 mol % hydrogen, based on a total number of moles of the off gas stream. In an embodiment, an amount of CO₂ present in the off gas stream is less than 10 mol %, less than 5 mol %, less than 3 mol %, or less than 1 mol %; greater than 0.01 mol %, greater than 0.05 mol %, greater than 0.1 mol %, or greater than 0.2 mol %; or a combination thereof, based on a total number of moles of the off gas stream. In an embodiment, C₂₋₄ hydrocarbons may be present in the off gas stream, for example, an amount of less than 10 mol %, less than 9 mol %, less than 7 mol %, or less than 5 mol %; greater than 0.01 mol %, greater than 0.1 mol %, greater than 0.5 mol %, or greater than 1 mol %; or a combination thereof, based on a total number of moles of the off gas stream.

In an embodiment, an off gas source (e.g., another chemical or molecule production system) is integrated with a methanol production assembly. Natural gas can be sent from the methanol production assembly to the off gas source.

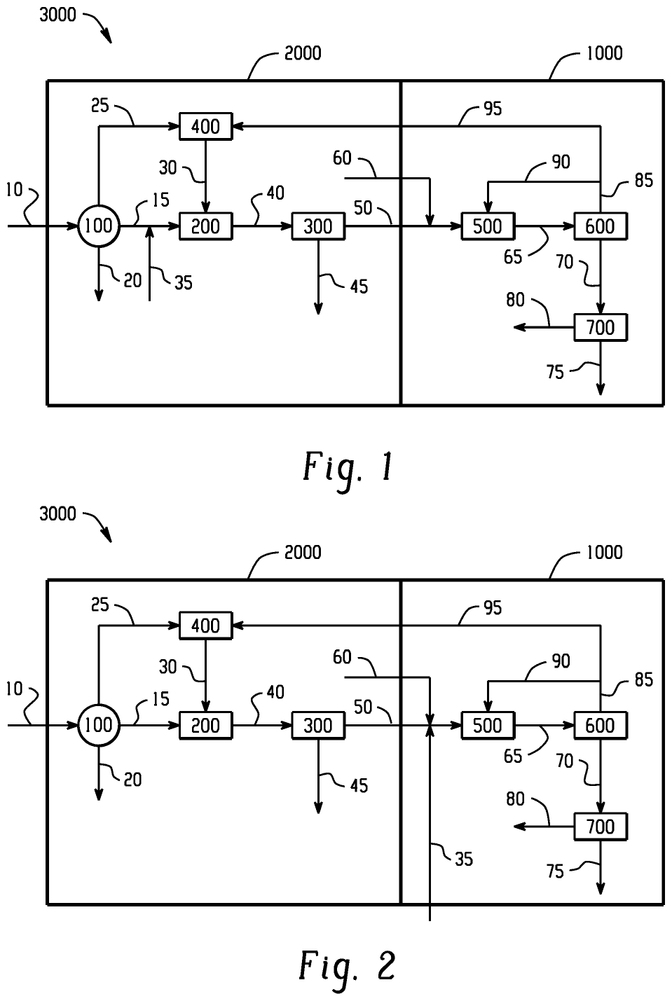

FIG. 1 is a diagram of an embodiment for utilizing off gas including hydrogen for methanol production. Methanol production assembly 3000 includes a synthesis portion 1000 and a reforming portion 2000. A natural gas feed stream 10 (including, for example, CH₄, C₂H₆, and C₃H₈) can be fed to the reforming portion 2000 of the methanol production assembly 3000, for example, a separator or splitter 100 of the reforming portion 2000 of the methanol production assembly 3000. The natural gas feed stream 10 can be separated into, for example, at least a reforming feed stream 15 including natural gas, and an auxiliary natural gas stream 20; and in an embodiment, at least a reforming feed stream 15 including natural gas, an auxiliary natural gas stream 20, and a natural gas fuel stream 25. The reforming feed stream 15 can be fed to a reforming reactor 200 to form a synthesis gas stream 50. In an embodiment, a reforming reactor product stream 40 can be subjected to cooling and separation 300, in which wastewater stream 45 and the synthesis gas stream 50 can be separated from the reforming reactor product stream 40. The natural gas fuel stream 25 can be fed to a heater (e.g., furnace) 400 to supply heat 30 to the reforming reactor 200 (the reforming reaction being endothermic). An off gas stream 35 including hydrogen can be combined with the reforming feed stream 15 prior to being fed into the reforming reactor 200. A purge gas stream 95 can also be fed to the heater 400.

The synthesis gas stream 50 and a CO₂ stream 60 are fed to the synthesis portion 1000 of the methanol production assembly 3000; for example, said streams may be fed to a methanol synthesis reactor 500 of the synthesis portion 1000 of the methanol production assembly 3000. The methanol synthesis reactor 500 produces a methanol synthesis reactor product stream 65, which can be subjected to cooling and separation 600. A cooled stream 70 from the cooling and separation 600 can be subjected to pressure reduction and separation 700. Product streams from the pressure reduction and separation 700 can include a crude methanol stream 75 and a vent gas stream 80. The crude methanol stream 75 can be separated and sent for further purification. A gas stream 85 from the cooling and separation 600 can be separated into a recycle stream 90, which can be recycled to the methanol synthesis reactor 500, and the purge gas stream 95.

FIG. 2 is a diagram of an embodiment for utilizing off gas including hydrogen for methanol production. Similar to FIG. 1, methanol production assembly 3000 includes a synthesis portion 1000 and a reforming portion 2000. A natural gas feed stream 10 (including, for example, CH₄, C₂H₆, and C₃H₈) can be fed to the reforming portion 2000 of the methanol production assembly 3000, for example, a separator or splitter 100 of the reforming portion 2000 of the methanol production assembly 3000. The natural gas feed stream 10 can be separated into, for example, at least a reforming feed stream 15 including natural gas, and an auxiliary natural gas stream 20; and in an embodiment, at least a reforming feed stream 15 including natural gas, an auxiliary natural gas stream 20, and a natural gas fuel stream 25. The reforming feed stream 15 can be fed to a reforming reactor 200 to form a synthesis gas stream 50. In an embodiment, a reforming reactor product stream 40 can be subjected to cooling and separation 300, in which wastewater stream 45 and the synthesis gas stream 50 can be separated from the reforming reactor product stream 40. The natural gas fuel stream 25 can be fed to a heater (e.g., furnace) 400 to supply heat 30 to the reforming reactor 200 (the reforming reaction being endothermic). A purge gas stream 95 can also be fed to the heater 400.

An off gas stream 35 including hydrogen, the synthesis gas stream 50, and a CO₂ stream 60 are fed to the synthesis portion 1000 of the methanol production assembly 3000; for example, said streams may be fed to a methanol synthesis reactor 500 of the synthesis portion 1000 of the methanol production assembly 3000. The methanol synthesis reactor 500 produces a methanol synthesis reactor product stream 65, which can be subjected to cooling and separation 600. A cooled stream 70 from the cooling and separation 600 can be subjected to pressure reduction and separation 700. Product streams from the pressure reduction and separation 700 can include a crude methanol stream 75 and a vent gas stream 80. The crude methanol stream 75 can be separated and sent for further purification. A gas stream 85 from the cooling and separation 600 can be separated into a recycle stream 90, which can be recycled to the methanol synthesis reactor 500, and the purge gas stream 95.

Compared to a method not utilizing off gas addition, in the disclosed method, a percentage of the reforming feed stream 15 relative to a sum of the total of the reforming feed stream 15 and natural gas fuel stream 25 can be increased, and a percentage of the natural gas fuel stream 25 relative to the sum of the total of the reforming feed stream 15 and natural gas fuel stream 25 can be decreased.

### Methanol Synthesis

A synthesis portion of a methanol production assembly can include one or more methanol synthesis reactors, one or more gas-liquid separators, one or more heat exchangers, one or more recycle streams, one or more steam drums, or a combination thereof. A process for producing methanol can include a step of feeding synthesis gas to the synthesis portion of a methanol production assembly. The synthesis portion of a methanol production assembly can be operable to produce a crude methanol stream and a purge gas stream. The synthesis portion of a methanol production assembly can include a methanol synthesis reactor operable to produce a methanol synthesis reactor effluent stream, which can include methanol, water, H₂, CO, CO₂, and hydrocarbons. The synthesis portion of a methanol production assembly can include any reactor suitable for a methanol synthesis reaction from CO and H₂, such as, for example, a trickle bed reactor, a fluidized bed reactor, a slurry reactor, a loop reactor, a cooled multi tubular reactor, or a combination thereof.

Methanol (CH₃OH) synthesis reactions include:

CO+2H₂═CH₃OH

CO₂+3H₂═CH₃OH+H₂O.

Methanol synthesis from CO, CO₂ and H₂ is a catalytic process, and can be conducted in the presence of copper based catalysts. The methanol synthesis reactor can include a methanol production catalyst, such as any suitable methanol synthesis catalyst. Nonlimiting examples of methanol production catalysts suitable for use in the methanol synthesis reactor of the synthesis portion of a methanol production assembly include Cu, Cu/ZnO, Cu/ThO₂, Cu/Zn/Al₂O₃, Cu/ZnO/Al₂O₃, Cu/Zr, or a combination thereof.

A synthesis gas ratio can be defined as:

(H₂—CO₂)/(CO+CO₂).

Desirably, the synthesis gas ratio to maximize methanol production is greater than 2, for example, greater than 2.1, greater than 2.2, or greater than 2.3. As the disclosed method feeds off gas and CO₂ in addition to synthesis gas to the methanol synthesis reactor, a makeup (e.g., synthesis gas ratio) of the synthesis gas stream 50 fed to the methanol synthesis reactor 500 can be adjusted to maximize methanol production in the methanol synthesis reactor 500. In an embodiment, a synthesis gas ratio of the synthesis gas stream 50 fed to the methanol synthesis reactor 500 is greater than 1.8, preferably greater than 2.

### Steam Methane Reforming (SMR)

In an embodiment, the reforming reactor 200 of FIG. 1 or FIG. 2 can be an SMR reactor. SMR reactions describe the catalytic reaction of methane (CH₄) and steam (H₂O) to form carbon monoxide (CO) and hydrogen (H₂). SMR reactions include:

CH₄+H₂O═CO+3H₂

CO₂+H₂═CO+H₂O.

Effluent from an SMR reactor can be produced by reacting, via an SMR reaction, an SMR reactant mixture including hydrocarbons and steam in the SMR reactor. The SMR reactor effluent includes hydrogen, carbon monoxide, carbon dioxide, water, and unreacted methane.

Steam reforming catalysts utilized in an SMR reactor can include any suitable available steam reforming catalyst such as nickel (Ni), rhodium (Rh), or a combination thereof as active metal(s) on alumina; or a combination thereof. The reforming reactor 200, such as an SMR reactor, can be characterized by a steam to carbon molar ratio in the SMR reactant mixture of greater than or equal to about 1.5:1, for example, greater than or equal to about 2:1, greater than or equal to about 2.5:1, greater than or equal to about 2.7:1, greater than or equal to about 3.0:1, or in a range of from about 1.5:1 to 3.5:1, from about 1.5:1 to 3:1, or from about 1.5:1 to about 2.5:1. The SMR reactor effluent can be characterized by a H₂:CO molar ratio of greater than or equal to about 2.5:1, for example, greater than or equal to about 2.7:1, or greater than or equal to about 2.9:1. The SMR reaction can yield a synthesis gas with a H₂:CO molar ratio having the theoretical stoichiometric limit of 3.0:1, subject to reactants undergoing side reactions at the conditions used for the SMR reaction.

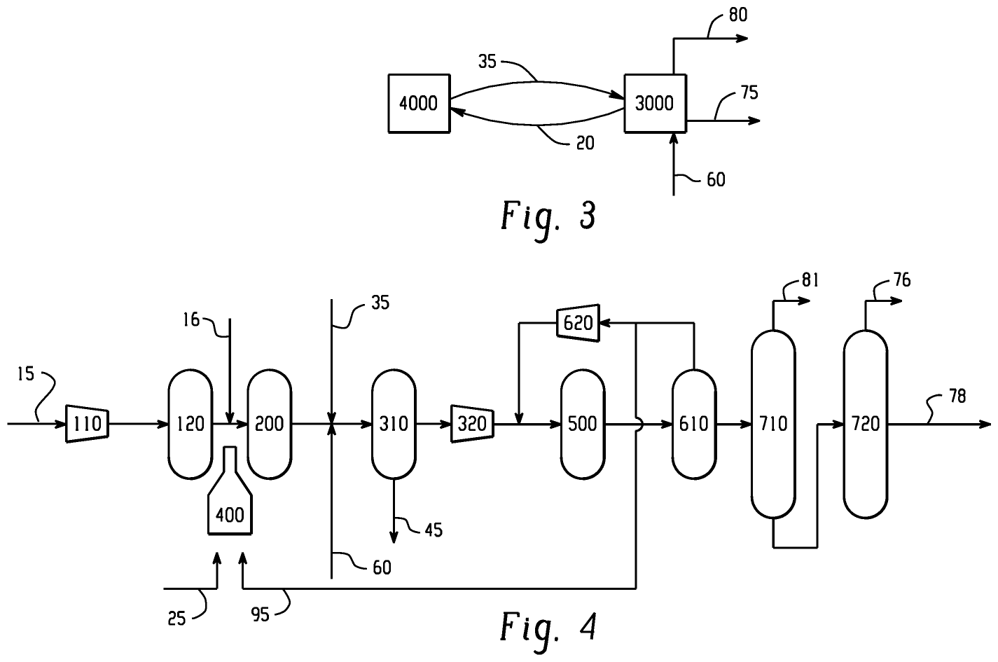

FIG. 3 is a diagram of an embodiment for utilizing CO₂ and off gas including hydrogen for methanol production. Off gas stream 35 and a CO₂ stream 60 are fed to a methanol production assembly 3000, which as depicted in FIG. 1 and FIG. 2 may include the synthesis portion 1000 and the reforming portion 2000. In an embodiment, off gas stream 35 and the CO₂ stream 60 can be fed to the synthesis portion 1000 of the methanol production assembly 3000. Products of the methanol production assembly 3000 can include the crude methanol stream 75 and the vent gas stream 80. The off gas stream 35 is supplied from an off gas source 4000. The auxiliary natural gas stream 20 is provided to the off gas source 4000 from the methanol production assembly 3000, for example, the reforming portion 2000 of the methanol production assembly 3000. Benefits of the disclosed method can include reduction in overall natural gas consumption (e.g., natural gas feed stream 10); reduction in CO₂ emissions from the methanol production assembly 3000 (e.g., in vent gas stream 80); increase in methanol production from the methanol production assembly 3000 (e.g., in crude methanol stream 75); potential NOx reduction; or a combination thereof.

The off gas source 4000 can be, for example, any suitable chemical or molecule (e.g., monomer or polymer) production system (e.g., a chemical plant) that produces an off gas including hydrogen. Examples of off gas sources 4000 include a methyl tert-butyl ether production system (e.g., which can include a paraffin (e.g., iso-butane) dehydrogenation system to provide iso-butene for the methyl tert-butyl ether production), an olefin production system, a chlor-alkali production system (e.g., a system in which chlorine and sodium hydroxide are produced simultaneously by electrolytic decomposition of sodium chloride), or a combination thereof.

This disclosure is further illustrated by the following examples, which are nonlimiting.

### EXAMPLES

FIG. 4 is a schematic overview of a process model simulated using Aspen Plus® V10 software. As seen in FIG. 4, reforming feed stream 15 including (purified) natural gas is fed to a natural gas compressor 110. A compressed natural gas stream exiting the natural gas compressor 110 is fed to saturator 120. A saturated stream exiting the saturator 120 and a steam stream 16 is fed to outlet of the saturator 120 before entering the (steam) reforming reactor 200. Synthesis gas formed in the (steam) reforming reactor 200 and CO₂ stream 60 are fed to a knockout 310, from which wastewater stream 45 is removed. A remaining stream exiting the knockout 310 is fed to synthesis gas compressor 320, and a compressed synthesis gas exiting the synthesis gas compressor 320 is fed to methanol synthesis reactor 500. A product stream exiting the methanol synthesis reactor 500 (e.g., equivalent to methanol synthesis reactor product stream 65 in FIG. 1 and FIG. 2) is fed to separator 610. A portion of a purge gas stream (e.g., equivalent to gas stream 85 in FIG. 1 and FIG. 2) withdrawn from the separator 610 is fed to recycle compressor 620. A compressed purge gas stream exiting the recycle compressor 620 is mixed with the compressed synthesis gas exiting the synthesis gas compressor 320 prior to being fed to methanol synthesis reactor 500. Another portion of the purge gas stream withdrawn from the separator 610 (shown as purge gas stream 95) and natural gas fuel stream 25 are fed to heater (e.g., furnace) 400, which supplies heat to the reforming reactor 200. In FIG. 4, an off gas stream 35 including hydrogen is fed with the synthesis gas formed in the (steam) reforming reactor 200 and the CO₂ stream 60 to the knockout 310.

A product stream exiting the separator 610 (e.g., equivalent to cooled stream 70 in FIG. 1 and FIG. 2) is fed to a distillation train including a topping column 710 and a refining column 720. A top product withdrawn from the topping column 710 is a light gas stream 81 (e.g., equivalent to vent gas stream 80 in FIG. 1 and FIG. 2) and a bottom product from the topping column 710 (e.g., equivalent to crude methanol stream 75 in FIG. 1 and FIG. 2) is fed to the refining column 720. A middle product of the refining column 720 is methanol stream 78. Top product 76 withdrawn from the refining column 720 includes methanol and water in vapor form.

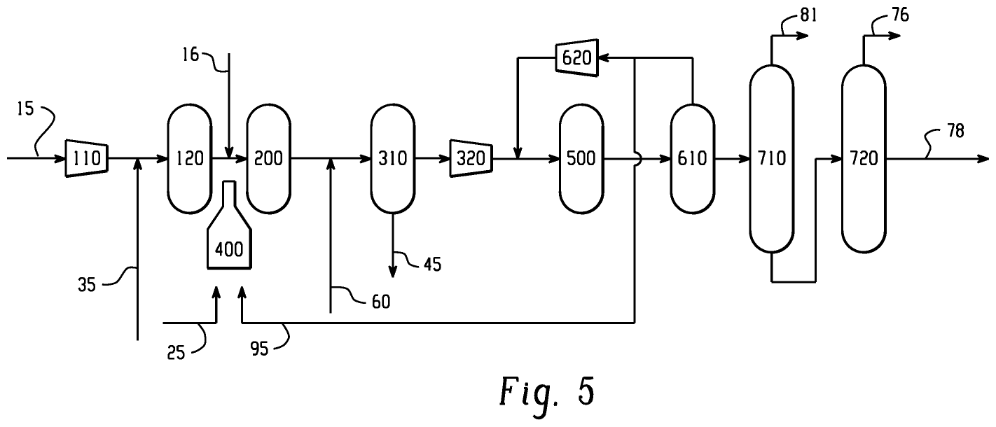

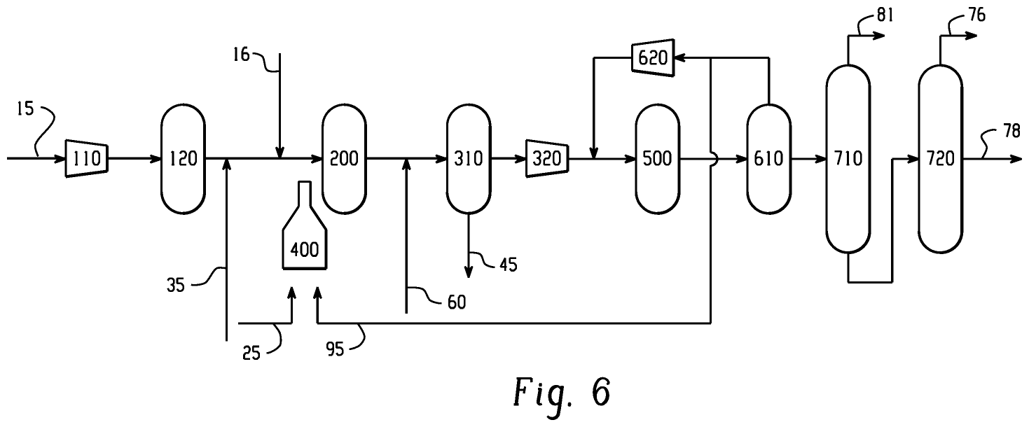

The examples provide a comparison of a scenario of no off gas addition with three different scenarios: 1) off gas stream addition in a synthesis portion of a methanol production assembly (shown in FIG. 4), 2) off gas stream addition in a reforming portion of the methanol production assembly prior to saturator 120 (shown in FIG. 5), and 3) off gas stream addition in the reforming portion of a methanol production assembly after saturator 120 (shown in FIG. 6). The composition of natural gas and off gas (in mole percent (mol %)) used in the examples are provided in Table 1.

TABLE 1

| | Natural gas | Off gas |
|---|---|---|
| Component | (mol %) | (mol %) |
| H₂ | 0.00 | 87.28 |
| CO | 0.00 | 1.29 |
| CO₂ | 0.21 | 0.96 |
| N₂ | 4.36 | 4.84 |
| CH₄ | 92.34 | 3.06 |
| Ethane | 2.34 | 0.20 |
| Propane | 0.50 | 0.73 |
| Butane | 0.17 | 0.19 |
| Pentane | 0.08 | 0.00 |
| Ethylene | 0.00 | 0.20 |
| Propylene | 0.00 | 1.16 |
| Butene | 0.00 | 0.08 |

Assumptions used in the simulations include: (1) a ratio (weight or volume) of an amount of natural gas fed to the reforming reactor to an amount of natural gas fed to the heater was varied to keep the bridge-wall temperature (e.g., the temperature of the flue gas at the entrance of a convection section after supplying required energy to the reforming reactor) constant; (2) the O₂ content in the flue gas was kept constant (1.1-1.2% volume % by dry basis); (3) a ratio (by volume) of steam to carbon to the reforming reactor was kept constant by adjusting process steam (steam stream 16 in FIG. 4) to the reforming reactor (200 in FIG. 4); (4) boil up ratios (the fractional amount of liquid that is boiled back into the column to the amount of liquid leaving) in the distillation columns were kept constant (0.38 for topping column and 6.1 for refining column); (5) recovery of methanol (methanol pure (quantity of methanol in stream 78 in FIG. 4)/methanol in crude (quantity of methanol in stream exiting separator 610 in FIG. 4)) was kept constant; (6) the recycle ratio (ratio of moles of vapor going out of methanol separator to the moles of make-up gas) for the three scenarios was kept the same as the scenario with no off gas addition; (7) equivalent additional CO₂ injected based on reaction stoichiometry of CO₂+3H₂═CH₃OH+H₂O; and (8) olefins and paraffins in the off gas were assumed to remain inert and not influence equilibrium reactions in the methanol synthesis reactor. The simulation results are provided in Table 2 (flows are in tons per hour (TPH)).

TABLE 2

| | | | Off gas | |
|---|---|---|---|---|
| | | | addition to | |
| | | | reforming | Off gas |
| | | | feed stream | addition in |
| | | Off gas | including | reforming |
| | No off | addition in | natural gas | portion |
| | gas | synthesis | (before | after |
| Parameters | addition | portion | saturator) | saturator |
| Crude methanol (TPH) | 112.9 | 117.1 | 115.5 | 115.8 |
| (Stream exiting separator 610 in FIG. 4) | | | | |
| Pure methanol (TPH) | 111.9 | 115.5 | 113.9 | 114.2 |
| (Stream 78 in FIG. 4) | | | | |
| Extra methanol (TPH) | 0.00 | 3.6 (115.5- | 2.0 (113.9- | 2.3 (114.2- |
| | | 111.9) | 111.9) | 111.9) |
| Synthesis gas ratio of stream exiting the | 2.4 | 2.3 | 2.3 | 2.3 |
| reforming reactor after CO₂ addition | | | | |
| ((H₂ − CO₂)/(CO + CO₂)) | | | | |
| Total natural gas for reforming reactor | 77 | 70 | 70 | 70 |
| (feed (stream 15) + fuel (stream 25)) | | | | |
| (TPH) | | | | |
| Natural gas to reforming reactor | 0.82 | 0.84 | 0.82 | 0.82 |
| feed/natural gas to reforming reactor fuel | | | | |
| (Stream 15 TPH/stream 25 TPH) | | | | |
| CO₂ addition (stream 60) (TPH) | 22.4 | 36.6 | 36.6 | 36.6 |
| Natural gas sent to methyl tert-butyl | 0.00 | 7 | 7 | 7 |
| ether production (TPH) | | | | |
| (Stream 20 in FIG. 1) | | | | |
| Off gas addition (stream 35) (TPH) | 0.00 | 6 | 6 | 6 |

The results of Table 2 show that if an off gas stream including hydrogen is added to either a reforming or synthesis portion of a methanol production assembly, more methanol can be produced.

Further results were obtained from the simulations. A comparison of the disclosed method versus a scenario of no off gas addition is provided in Table 3.

TABLE 3

| | No off | Off gas addition in |
|---|---|---|
| | gas | synthesis portion or |
| Operating Parameter | addition | reforming portion |
| Methanol production | X | 1.02X to 1.04X |
| (tons per hour) | | |
| Synthesis Gas Ratio of stream entering the | X | 0.9X to 1.0X |
| methanol synthesis reactor | | |
| (H₂ − CO₂/CO + CO₂) | | |
| Specific natural gas feed consumption | X | 0.9X to 1.0X |
| (One million British Thermal Units | | |
| (MMBTU)/ton of methanol) | | |

Based on the results of Table 3, utilizing CO₂ and an off gas stream including hydrogen is advantageous in terms of additional methanol production and overall natural gas consumption reduction.

This disclosure further encompasses the following aspects.

Aspect 1. A method for producing methanol, the method comprising: feeding a synthesis gas stream to a methanol production assembly; feeding an off gas stream comprising hydrogen to the methanol production assembly; feeding a CO₂ stream to the methanol production assembly; and reacting the synthesis gas stream and the CO₂ stream in the methanol production assembly to produce methanol, wherein the method further comprises reacting the off gas stream with the synthesis gas stream and the CO₂ stream in the methanol production assembly to produce methanol, or feeding a reforming feed stream and the off gas stream to a reforming portion of the methanol production assembly to form the synthesis gas stream.

Aspect 2. The method of Aspect 1, comprising reacting the off gas stream with the synthesis gas stream and the CO₂ stream in the methanol production assembly to produce methanol.

Aspect 3. The method of Aspect 1 or 2, comprising feeding the reforming feed stream and the off gas stream to the reforming portion of the methanol production assembly to form the synthesis gas stream.

Aspect 4. The method of any of the preceding aspects, wherein the off gas stream comprises 70 to less than 100 mole percent hydrogen, preferably 70 to 99 mole percent hydrogen, 70 to 95 mole percent hydrogen, 70 to 90 mole percent hydrogen, or 80 to 90 mole percent hydrogen, based on a total number of moles of the off gas stream.

Aspect 5. The method of any of the preceding aspects, wherein the off gas stream is generated during production of methyl tert-butyl ether.

Aspect 6. The method of any of Aspects 1-4, wherein the off gas stream is generated during production of olefins.

Aspect 7. The method of Aspect 2, further comprising adjusting an amount of the synthesis gas stream, the off gas stream, the CO₂ stream, or a combination thereof fed to the methanol production assembly.

Aspect 8. The method of Aspect 7, wherein after adjusting the amount of the synthesis gas stream, the off gas stream, the CO₂ stream, or the combination thereof fed to the methanol production assembly, a molar amount of H₂ minus CO₂ divided by a molar amount of CO plus CO₂ in the synthesis gas stream is greater than 1.8, preferably greater than 2.

Aspect 9. An integrated method for producing methanol, the method comprising: feeding a synthesis gas stream to a synthesis portion of a methanol production assembly; feeding an off gas stream comprising hydrogen from an off gas source to the methanol production assembly; feeding a CO₂ stream to the synthesis portion of the methanol production assembly; providing an auxiliary natural gas stream from a reforming portion of the methanol production assembly to the off gas source; and reacting the synthesis gas stream and the CO₂ stream in the synthesis portion of the methanol production assembly to produce methanol, wherein the method further comprises reacting the off gas stream with the synthesis gas stream and the CO₂ stream in the synthesis portion of the methanol production assembly to produce methanol, or feeding a reforming feed stream and the off gas stream to the reforming portion of the methanol production assembly.

Aspect 10. The method of Aspect 9, further comprising: feeding a natural gas feed stream to the reforming portion of the methanol production assembly; separating the natural feed gas stream into at least the reforming feed stream and the auxiliary natural gas stream; forming the synthesis gas stream from the reforming feed stream; and providing the auxiliary natural gas stream to the off gas source.

Aspect 11. The method of Aspect 9, further comprising: feeding a natural gas feed stream to the reforming portion of the methanol production assembly; separating the natural gas feed stream into at least the reforming feed stream, a natural gas fuel stream, and the auxiliary natural gas stream; feeding the reforming feed stream to a reforming reactor to form the synthesis gas stream; using the natural gas fuel stream as fuel to provide heat to the reforming reactor; and providing the auxiliary natural gas stream to the off gas source.

Aspect 12. The method of any of Aspects 9-11, wherein the off gas stream comprises 70 to less than 100 mole percent hydrogen, preferably 70 to 99 mole percent hydrogen, 70 to 95 mole percent hydrogen, 70 to 90 mole percent hydrogen, or 80 to 90 mole percent hydrogen, based on a total number of moles of the off gas stream.

Aspect 13. The method of any of Aspects 9-12, wherein the off gas source is a methyl tert-butyl ether production system.

Aspect 14. The method of any of Aspects 9-12, wherein the off gas source is a chemical or molecule production system.

Aspect 15. An integrated method for producing methanol, the method comprising: feeding a natural gas feed stream to a reforming portion of a methanol production assembly; separating the natural gas feed stream into at least a reforming feed stream, a natural gas fuel stream, and an auxiliary natural gas stream; feeding the reforming feed stream to a reforming reactor to form a synthesis gas stream; using the natural gas fuel stream as fuel to provide heat to the reforming reactor; providing the auxiliary natural gas stream to a chemical or molecule production system to produce a chemical or molecule; forming an off gas stream comprising hydrogen in the chemical or molecule production system; feeding the synthesis gas stream to a synthesis portion of the methanol production assembly; feeding the off gas stream from the chemical or molecule production system to the methanol production assembly; feeding a CO₂ stream to the synthesis portion of the methanol production assembly; and reacting the synthesis gas stream and the CO₂ stream in the synthesis portion of the methanol production assembly to produce methanol, wherein the method further comprises reacting the off gas stream with the synthesis gas stream and the CO₂ stream in the synthesis portion of the methanol production assembly to produce methanol, or feeding the off gas stream from the chemical or molecule production system to the reforming reactor in which synthesis gas stream is formed.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt %, or, more specifically, 5 wt % to 20 wt %”, is inclusive of the endpoints and all intermediate values of the ranges of “5 wt % to 25 wt %,” etc.). “Combinations” is inclusive of blends, mixtures, alloys, reaction products, and the like. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

Although the processes and methods of the present disclosure have been described with reference to exemplary embodiments thereof, the present disclosure is not limited to such exemplary embodiments and/or implementations. Rather, the processes and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A method for producing methanol, the method comprising:
feeding a synthesis gas stream to a methanol production assembly;
feeding an off gas stream comprising hydrogen to the methanol production assembly;
feeding a CO₂ stream to the methanol production assembly; and
reacting the synthesis gas stream and the CO₂ stream in the methanol production assembly to produce methanol,
wherein the method further comprises
reacting the off gas stream with the synthesis gas stream and the CO₂ stream in the methanol production assembly to produce methanol, or
feeding a reforming feed stream and the off gas stream to a reforming portion of the methanol production assembly to form the synthesis gas stream.

2. The method of claim 1, comprising reacting the off gas stream with the synthesis gas stream and the CO₂ stream in the methanol production assembly to produce methanol.

3. The method of claim 1, comprising feeding the reforming feed stream and the off gas stream to the reforming portion of the methanol production assembly to form the synthesis gas stream.

4. The method of claim 1, wherein the off gas stream comprises 70 to less than 100 mole percent hydrogen, based on a total number of moles of the off gas stream.

5. The method of claim 1, wherein the off gas stream is generated during production of methyl tert-butyl ether.

6. The method of claim 1, wherein the off gas stream is generated during production of olefins.

7. The method of claim 2, further comprising adjusting an amount of the synthesis gas stream, the off gas stream, the CO₂ stream, or a combination thereof fed to the methanol production assembly.

8. The method of claim 7, wherein after adjusting the amount of the synthesis gas stream, the off gas stream, the CO₂ stream, or the combination thereof fed to the methanol production assembly, a molar amount of H₂ minus CO₂ divided by a molar amount of CO plus CO₂ in the synthesis gas stream is greater than 1.8.

9. An integrated method for producing methanol, the method comprising:
feeding a synthesis gas stream to a synthesis portion of a methanol production assembly;
feeding an off gas stream comprising hydrogen from an off gas source to the methanol production assembly;
feeding a CO₂ stream to the synthesis portion of the methanol production assembly;
providing an auxiliary natural gas stream from a reforming portion of the methanol production assembly to the off gas source; and
reacting the synthesis gas stream and the CO₂ stream in the synthesis portion of the methanol production assembly to produce methanol,
wherein the method further comprises
reacting the off gas stream with the synthesis gas stream and the CO₂ stream in the synthesis portion of the methanol production assembly to produce methanol, or
feeding a reforming feed stream and the off gas stream to the reforming portion of the methanol production assembly.

10. The method of claim 9, further comprising:
feeding a natural gas feed stream to the reforming portion of the methanol production assembly;
separating the natural feed gas stream into at least the reforming feed stream and the auxiliary natural gas stream;
forming the synthesis gas stream from the reforming feed stream; and
providing the auxiliary natural gas stream to the off gas source.

11. The method of claim 9, further comprising:
feeding a natural gas feed stream to the reforming portion of the methanol production assembly;
separating the natural gas feed stream into at least the reforming feed stream, a natural gas fuel stream, and the auxiliary natural gas stream;
feeding the reforming feed stream to a reforming reactor to form the synthesis gas stream;
using the natural gas fuel stream as fuel to provide heat to the reforming reactor; and
providing the auxiliary natural gas stream to the off gas source.

12. The method of claim 1, wherein the off gas stream comprises 70 to less than 100 mole percent hydrogen, 70 to 99 mole percent hydrogen, 70 to 95 mole percent hydrogen, based on a total number of moles of the off gas stream.

13. The method of claim 1, wherein the off gas source is a methyl tert-butyl ether production system.

14. The method of claim 1, wherein the off gas source is a chemical or molecule production system.

15. An integrated method for producing methanol, the method comprising:
feeding a natural gas feed stream to a reforming portion of a methanol production assembly;
separating the natural gas feed stream into at least a reforming feed stream, a natural gas fuel stream, and an auxiliary natural gas stream;
feeding the reforming feed stream to a reforming reactor to form a synthesis gas stream;
using the natural gas fuel stream as fuel to provide heat to the reforming reactor;
providing the auxiliary natural gas stream to a chemical or molecule production system to produce a chemical or molecule;
forming an off gas stream comprising hydrogen in the chemical or molecule production system;
feeding the synthesis gas stream to a synthesis portion of the methanol production assembly;
feeding the off gas stream from the chemical or molecule production system to the methanol production assembly;
feeding a CO₂ stream to the synthesis portion of the methanol production assembly; and
reacting the synthesis gas stream and the CO₂ stream in the synthesis portion of the methanol production assembly to produce methanol,
wherein the method further comprises
reacting the off gas stream with the synthesis gas stream and the CO₂ stream in the synthesis portion of the methanol production assembly to produce methanol, or
feeding the off gas stream from the chemical or molecule production system to the reforming reactor in which synthesis gas stream is formed.
